(19) United States
(12) Patent Application Publication
(10) Pub. No.: US 2021/0244500 A1
(43) Pub. Date: Aug. 12, 2021

(54) Method of Neuromuscular Therapy

(71) Applicant: Neil Sheehy, Minneapolis, MN (US)

(72) Inventor: Neil Sheehy, Minneapolis, MN (US)

(21) Appl. No.: 16/786,195

(22) Filed: Feb. 10, 2020

(51) Int. Cl. A61C 5/00; A61C 7/08

(52) U.S. Cl. CPC A61C 5/007; A61C 7/08

(57) ABSTRACT

A method of neuromuscular therapy. The method of neuromuscular therapy has the steps of: opening a mouth of a patient; assessing a plurality of contours of a hard palate of the patient; engaging the hard palate of the patient at a first desired location; applying gradual pressure to the first desired location until the hard palate begins to move; and applying constant pressure to the first desired location until the hard palate is repositioned to a desired orientation. The method of neuromuscular therapy can be used to increase oxygenation in the patient's blood as well as restoring neuromuscular balance throughout the patient's body.

### BACKGROUND OF THE INVENTION

The present invention relates to neuromuscular therapy. More particularly, the present therapy provides for a method of neuromuscular therapy involving manipulation of the patient's hard palate. In some embodiments, a custom retainer device is utilized to hold and maintain the hard and soft palates in a desired orientation.

Neuromuscular therapy is the controlled manipulation of the body's soft tissues that aims to treat various ailments including chronic pain involving the muscular and nervous systems. Many people's bodies slowly decline over the course of their lives. These people's bodies are not able to deliver what their mind wants them to do. This is caused by a growing imbalance in the body's neurochemical systems. Restoration of these systems into balance relieves the ailments and rejuvenates the body. By treating the body through neuromuscular therapy, circulation improves, nerves decompress, postural issues are alleviated, and injuries caused by repetitive movement are assuaged.

Devices have been disclosed in the known art that relate to treating chronic pain as well as restoring balance to the body's neurochemical systems. These include devices that have been patented and disclosed in patent application publications. However, the devices in the known art have several drawbacks. Medications, drugs, and pills typically only target the symptoms of the ailment and do not delve down to the underlying cause. These medications are quite costly and can be prohibitively expensive if not covered by insurance. Additionally, many medications are habit forming and necessitate that the patient remain on the medication for extended periods of time.

Various forms of massage, similarly, may serve to temporarily alleviate the symptoms of the ailment. However, such treatment is typically short term and symptoms can begin to reemerge shortly thereafter. Many forms of massage are also guided by the patient and do not target key centers and pressure points in the body giving rise to the ailment. Additionally, many forms of massage rely on mechanical devices which are inexact and do not provide precise control over the body's soft tissues. These devices are not always at hand and are typically owned by the massage therapist. Because massage, and other forms of similar treatment, are only effective for a short time, the patient must rely on seeing the therapist often in order to treat chronic symptoms.

The present invention substantially diverges in design elements from the known art and consequently it is clear that there is a need in the art for an improvement to treating chronic pain as well as restoring balance to the body's neurochemical systems. In this regard the present invention substantially fulfills these needs.

### SUMMARY OF THE INVENTION

In view of the foregoing disadvantages inherent in the known types of medications and therapies now present in the prior art, the present invention provides a method of neuromuscular therapy which can be utilized to increase oxygenation in the patient's blood as well as restoring neuromuscular balance throughout the patient's body. The present method of neuromuscular therapy comprises the steps of: opening a mouth of a patient; assessing a plurality of contours of a hard palate of the patient; engaging the hard palate of the patient at a first desired location; applying gradual pressure to the first desired location until the hard palate begins to move; and applying constant pressure to the first desired location until the hard palate is repositioned to a desired orientation.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a cross-sectional view of a representative head of a patient, with a focus on the hard and soft palates.

FIG. 2 shows a bottom-up view of a representative mouth of a patient, and an embodiment of the custom retainer, in use.

FIG. 3 shows a flow chart of an embodiment of the method of neuromuscular therapy.

FIG. 4 shows a flow chart of an embodiment of the method of manufacturing a retainer for neuromuscular therapy.

FIG. 5 shows a flow chart of an embodiment of the method of use of a retainer for neuromuscular therapy.

### DETAILED DESCRIPTION OF THE INVENTION

Reference is made herein to the attached drawings. Like reference numerals are used throughout the drawings to depict like or similar elements of the method of neuromuscular therapy. For the purposes of presenting a brief and clear description of the present invention, a preferred embodiment will be discussed as used for the method of neuromuscular therapy. The figures are intended for representative purposes only and should not be considered to be limiting in any respect.

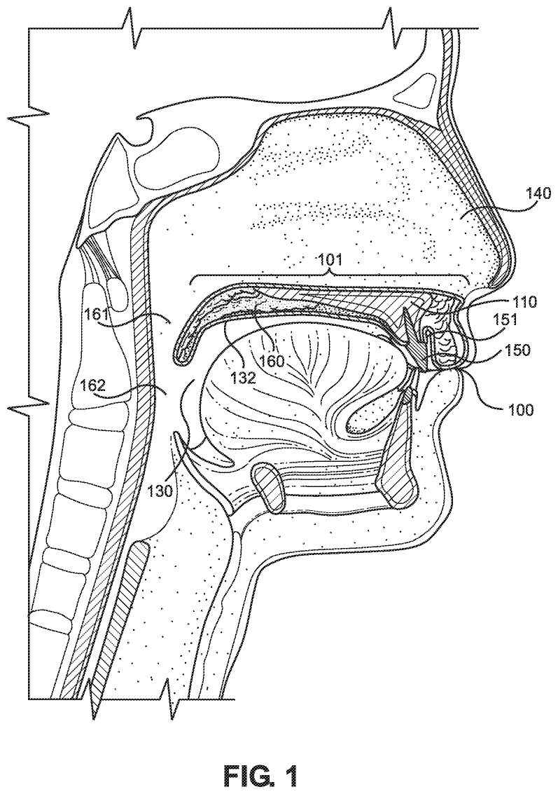

Referring now to FIG. 1, there is shown a cross-sectional view of a representative head of a patient, with a focus on the hard and soft palates. The anatomy of a patient's head comprises a palate 101. The palate 101 is a structure that forms a division between the oral cavity 130 and a nasal cavity 140 of the patient's head.

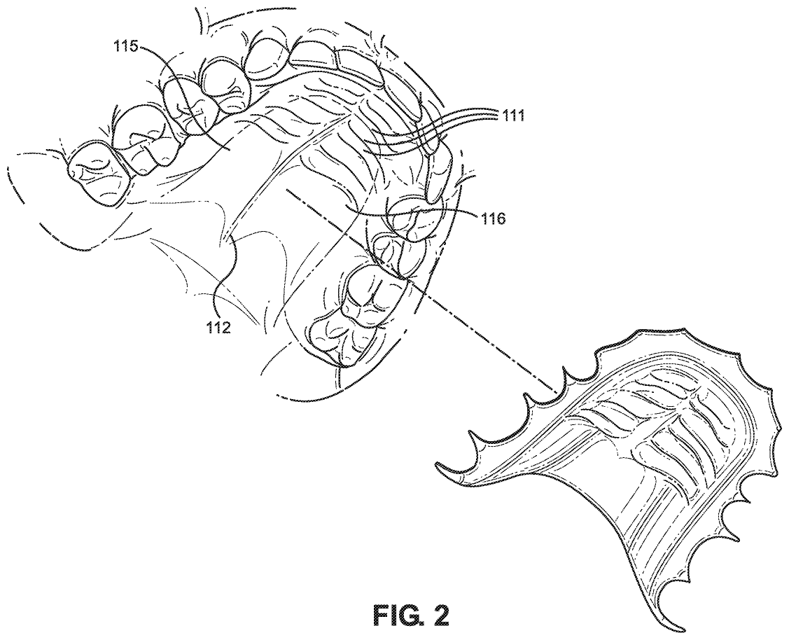

The hard palate 110 comprises a bony plate. The portion of the hard palate 110 above the bony plate is covered by respiratory mucosa and forms the floor of the nasal cavity 140. The portion of the hard palate 110 below the bony plate is covered by a layer of oral mucosa and forms a portion of the roof of the patient's mouth 100. The oral mucosa of the hard palate 110 includes a plurality of transverse palatine folds (see FIG. 2, 111). These transverse palatine folds are unique to a patient's mouth and can be used as a landmark. A plurality of teeth 150 of the patient are embedded in an anterior portion 151 of the hard palate 110 in a semi-circular formation. Posteriorly, the hard palate 110 is continuous with the soft palate 160. The primary functions of the hard palate 110 include feeding and speech, but as the hard palate 110 also defines the floor of the nasal cavity 140, the hard palate 110 has an effect on the respiratory system of the body which controls oxygenation of the blood. By manipulation of the orientation and location of the hard palate 110, the dimensions of the nasal cavity 140 are affected and the flow of air in the nasal cavity 140 can be altered and adjusted.

The soft palate 160 is the muscular portion of the palate 101 situated in the posterior portion 132 of the roof 102 of the patient's mouth. The soft palate 160 is a movable muscle fold suspended from a posterior portion of the hard palate 110. The soft palate 160 separates the nasopharynx 161 from the oropharynx 162. The soft palate 160 performs a number of functions including isolating the mouth 100 from the oropharynx 162 during eating. The soft palate 160 also has a role in the closure of the pharyngeal isthmus to adjust the voice and plays a role in coughing and sneezing. As the soft palate 160 is continuous with the hard palate 110, manipulation of one results in the movement in the other. Thus, manipulation of the orientation of the hard palate 110 results in movement of the soft palate 160, and vice versa.

Referring now to FIG. 2, there is shown a bottom-up view of a representative mouth of a patient, and an embodiment of the custom retainer, in use. A mid-palatine raphe 112 is a median longitudinal ridge that divides a left side 115 of the hard palate 110 from a right side 116 of the hard palate 110. In patients with cleft palate, the left side 115 and the right side 116 are separated, while in healthy adults, the left side 115 and the right side 116 are joined. The structure of the palate in each patient is unique and various landmarks such as the mid-palatine raphe 112 help to assess the orientation of the palate relative to the nasal cavity. Other defining landmarks can include the contours and folds of the oral mucosa and the transverse palatine folds 111.

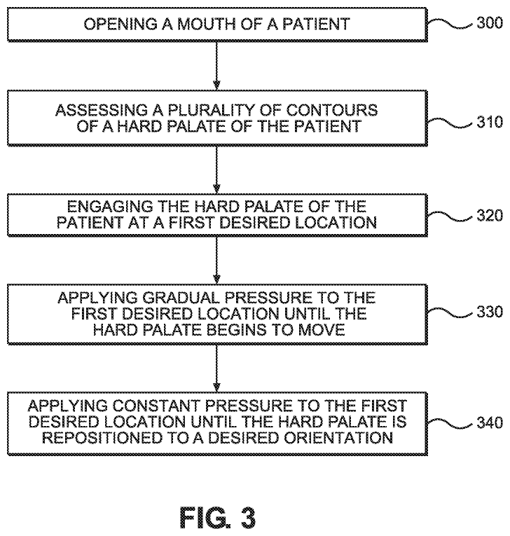

Referring now to FIG. 3, there is shown a flow chart of an embodiment of the method of neuromuscular therapy. In the shown embodiment, the method of neuromuscular therapy comprises the first step of opening a mouth of a patient 300. One of ordinary skill in the art will understand that the patient can be directed to opening their mouth on their own, however a neuromuscular therapist or device may be used to gain access to the interior of the patient's mouth. For the sake of simplicity, a neuromuscular therapist will be used for the remainder of this disclosure. However, this disclosure is not limited to a neuromuscular therapist accomplishing each step. One of ordinary skill in the art will understand that various embodiments of the method of neuromuscular therapy may utilize a combination of the patient, a neuromuscular therapist, and various devices. Such outside force may be necessary where the patient is sedated, for example. Assessment of a plurality of contours of a hard palate of the patient 310 allows the neuromuscular therapist to get acclimated with the shape and positioning of the hard palate in relation to the oral and nasal cavities. The relative positioning of each patient's head and mouth anatomy are unique and differs from one patient to another. Thus, identifying landmarks and relative positioning between the landmarks aids is planning the course of therapy and treatment in the patient. In one embodiment, this assessment occurs by applying a probing pressure to various landmarks on the hard palate. Such a probing pressure is a pressure that is not enough to move the hard palate but enables the neuromuscular therapist to identify various landmarks by touch and the resistance various parts of the hard palate provide to such pressure.

Once assessment of the plurality of contours of the hard palate of the patient 310 is made, the neuromuscular therapist engages the hard palate at a first desired location 320. Such first desired location is determined by the neuromuscular therapist as a result of the assessment 310. In one embodiment, the first desired location is at a midline of the hard palate. In a further embodiment, the first desired location is at a point along the mid-palatine raphe. Gradual pressure is applied to the first desired location until the hard palate begins to move 330. In one embodiment, the gradual pressure is applied via a hand and fingers of the neuromuscular therapist. In another embodiment, the gradual pressure is applied via a device such as a pressure probe. Gradual pressure is utilized so as to minimize the discomfort of the patient during the procedure. In one embodiment, the pressure begins at a very low level and the neuromuscular therapist allows the soft tissue surrounding the first desired location to relax and envelop the source of pressure. Thus, one of ordinary skill in the art will understand that the gradual pressure may include periods of maintaining or releasing a force on the first desired location while such relaxation and envelopment develops. Once the hard palate begins to move, constant pressure is applied to the first desired location until the hard palate is repositioned to a desired orientation 340. In one embodiment, the constant pressure is applied in a trajectory towards a nasal cavity of the patient. One of ordinary skill in the art will further understand that such constant pressure may be intermixed with increasing or decreasing force in order to maintain a constant pressure on the first desired location.

In one embodiment, the neuromuscular therapist utilizes a second desired location of the hard palate by engaging the hard palate of the patient at such a second desired location, applying gradual pressure to the second desired location until the hard palate begins to move, and applying constant pressure to the second desired location. In a further embodiment, additional new desired locations of the hard palate are utilized by repeating the steps of engaging new desired locations of the hard palate, applying gradual pressure to the new desired locations until the new desired locations begin to move, and applying constant pressure to the new desired locations until the hard palate is repositioned to the desired location. By engaging and applying pressure to multiple desired locations of the hard palate, the neuromuscular therapist is able to obtain finer control of the movement of the hard palate and is better able to reposition the hard palate to a desired location.

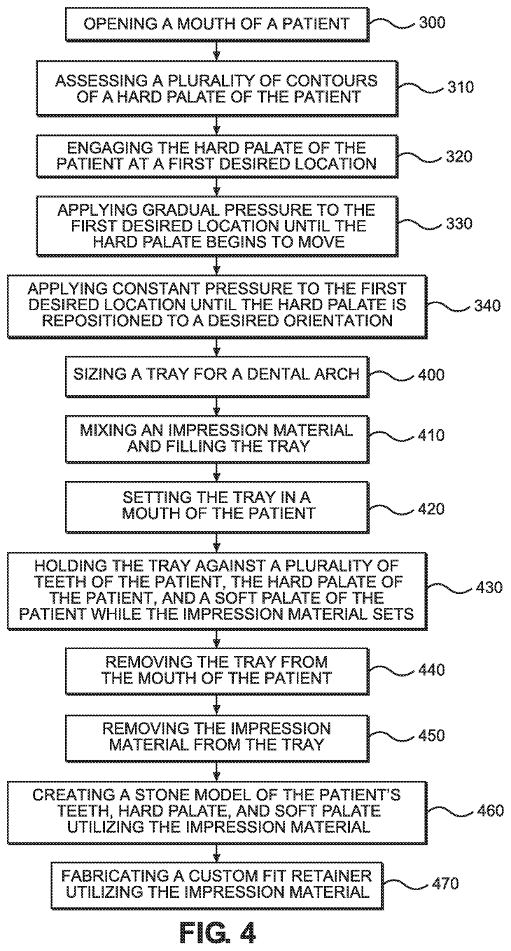

Referring now to FIG. 4, there is shown a flow chart of an embodiment of the method of manufacturing a retainer for neuromuscular therapy. Some patients respond very well to neuromuscular therapy after one session. However, over time some patients regress and find that the original symptoms and ailments begin to reemerge. Recurrent therapy sessions are one option to stem this regression. Some patients are unable to attend multiple therapy sessions because of financial or scheduling reasons. A custom retainer is able to be manufactured for patients that will also aid in stemming this regression. The method of manufacturing a retainer for neuromuscular therapy begins in the same manner as the method of neuromuscular therapy 300, 310, 320, 330, and 340 as detailed in FIG. 3, above. This is because the hard and soft palates must first be positioned into the desired orientation prior to maintaining said position with a custom retainer. Thus, the method for manufacturing a retainer for neuromuscular therapy progresses through the same steps as the method of neuromuscular therapy detailed in the discussion of FIG. 3, above.

The method for manufacturing a retainer for neuromuscular therapy continues by sizing a tray for a dental arch 400. The tray for the dental arch must be the appropriate size for not only the patient's teeth, but also to capture the unique contours of the roof of the patient's mouth. An impression material is mixed and the tray is filled with such material 410. The tray is then set in the patient's mouth 420 and held against the patient's teeth, hard palate, and soft palate while the impression material sets 430. By holding such impression material against the teeth, hard palate, and soft palate, the contours of each part of the patient's anatomy are captured by the impression material. Thus, the desired orientation of the hard and soft palates is preserved. Once the impression material is set, the tray is removed from the mouth of the patient 440 and the impression material is removed from the tray 450. A stone model of the patient's teeth, hard palate, and soft palate is made utilizing the impression material 460. In this manner, the contours of each part of the patient's anatomy is transferred to the stone model. This stone model is then utilized to create a custom fit retainer 470. One of ordinary skill in the art will understand how to create a custom fit retainer that holds the hard and soft palates of the patient in the desired orientation by use of the stone models. The creation of this custom fit retainer can include sending the stone model to an orthodontic laboratory to create such a retainer. Unlike some retainers which removably secure to the patient's teeth by mechanisms such as clips, the present retainer does not utilize wires, clips, or other fasteners, but rather relies on the vacuum created between the roof of the patient's mouth and the contours of the hard and soft palate, which are mirrored in the retainer, to hold the retainer in place.

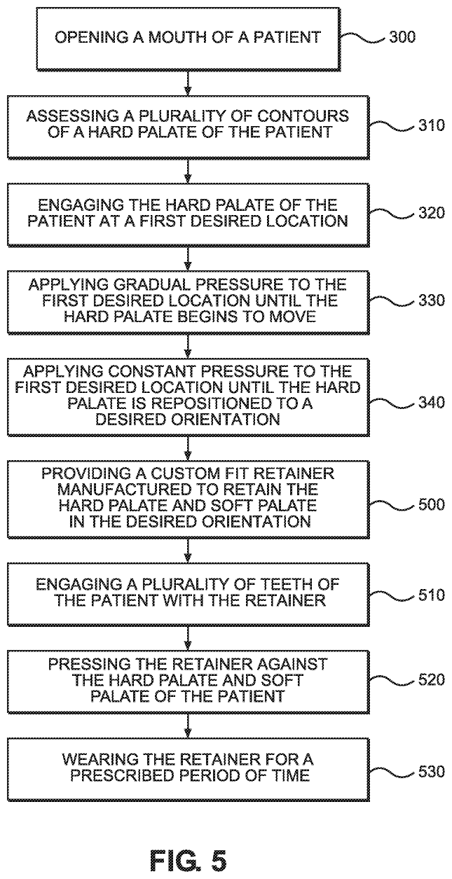

Referring now to FIG. 5, there is shown a flow chart of an embodiment of the method of use of a retainer for neuromuscular therapy. The method of use of a retainer for neuromuscular therapy begins in the same manner as the method of neuromuscular therapy 300, 310, 320, 330, and 340 as detailed in FIG. 3, above. Once the custom retainer is manufactured it can be used to maintain the desired orientation of the hard and soft palates. Use of the retainer comprises the steps of providing a custom fit retainer manufactured to retain the hard palate and soft palate in the desired orientation 500. The retainer engages a plurality of teeth of the patient 510. In one embodiment, the retainer includes clips that are utilized to fasten around several of the patient's teeth. In a further embodiment, the clips removably secure to the front teeth. In another embodiment, the clips secure to at least one molar. Once the retainer is engaged with the plurality of teeth, the retainer is pressed against the hard palate and soft palate of the patient 520. By pressing the retainer against the hard and soft palate, the contours of the hard and soft palate receive the complementary contours of the retainer that were imprinted on the retainer through the manufacturing process described above. The patient wears the retainer for a prescribed period of time 530. In one embodiment, the prescribed period of time is determined by the neuromuscular therapist. Patients may choose to wear the retainer at all times to maintain such orientation. However, some patients may decide to only wear the retainer at selected times.

Some patients who do not wear the retainer at all times may find that the hard and soft palate begin to regress and deviate from the desired orientation. Through repeat neuromuscular therapy sessions, or repeated use of the retainer, the patient's anatomy will begin to maintain the desired orientation on its own. For those patients who begin to regress, further neuromuscular therapy sessions coupled with use of the retainer will restore the hard and soft palates to the desired orientation. The method of use of a retainer for neuromuscular therapy comprises the steps of the original neuromuscular therapy, couple with the steps of use of the retainer to maintain the orientation.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A method of neuromuscular therapy, comprising the steps of:
opening a mouth of a patient;
assessing a plurality of contours of a hard palate of the patient;
engaging the hard palate of the patient at a first desired location;
applying gradual pressure to the first desired location until the hard palate begins to move; and
applying constant pressure to the first desired location until the hard palate is repositioned to a desired orientation.

2) The method of neuromuscular therapy of claim 1, wherein the constant pressure is in a trajectory towards a nasal cavity of the patient.

3) The method of neuromuscular therapy of claim 1, further comprising the steps of:
engaging the hard palate of the patient at a second desired location of the hard palate;
applying gradual pressure to the second desired location until the hard palate begins to move; and
applying constant pressure to the second desired location.

4) The method of neuromuscular therapy of claim 3, further comprising the steps of:
repeating the steps of engaging new desired locations of the hard palate, applying gradual pressure to the new desired locations until the new desired locations begin to move, and applying constant pressure to the new desired locations until the hard palate is repositioned to a desired orientation.

5) The method of neuromuscular therapy of claim 1, wherein the first desired location is at a midline of the hard palate.

6) The method of neuromuscular therapy of claim 1, wherein the assessment of the contours of the patient's hard palate is achieved by applying a probing pressure to landmarks on the hard palate.

7) A method of manufacturing a retainer for neuromuscular therapy, comprising the steps of:
opening a mouth of a patient;
assessing a plurality of contours of a hard palate of the patient;
engaging the hard palate of the patient at a first desired location;
applying gradual pressure to the first desired location until the hard palate begins to move;
applying constant pressure to the first desired location until the hard palate is repositioned to a desired orientation;
sizing a tray for a dental arch;
mixing an impression material and filling the tray;
setting the tray in a mouth of the patient;
holding the tray against a plurality of teeth of the patient, the hard palate of the patient, and a soft palate of the patient while the impression material sets;
removing the tray from the mouth of the patient;
removing the impression material from the tray;
creating a stone model of the patient's teeth, hard palate, and soft palate utilizing the impression material; and
fabricating a custom fit retainer utilizing the impression material.

8) The method of neuromuscular therapy of claim 7, wherein the constant pressure is in a trajectory towards a nasal cavity of the patient.

9) The method of neuromuscular therapy of claim 7, further comprising the steps of:
engaging the hard palate of the patient at a second desired location of the hard palate;
applying gradual pressure to the second desired location until the hard palate begins to move; and
applying constant pressure to the second desired location.

10) The method of neuromuscular therapy of claim 9, further comprising the steps of:
repeating the steps of engaging new desired locations of the hard palate, applying gradual pressure to the new desired locations until the new desired locations begin to move, and applying constant pressure to the new desired locations until the hard palate is repositioned to a desired orientation.

11) The method of neuromuscular therapy of claim 7, wherein the first desired location is at a midline of the hard palate.

12) The method of neuromuscular therapy of claim 7, wherein the assessment of the contours of the patient's hard palate is achieved by applying a probing pressure to landmarks on the hard palate.

13) The method of neuromuscular therapy of claim 7, further comprising the steps of:
engaging a plurality of teeth of the patient with the retainer;
pressing the retainer against the hard palate and soft palate of the patient; and
wearing the retainer for a prescribed period of time.

14) A method of use of a retainer for neuromuscular therapy, comprising the steps of:
opening a mouth of a patient;
assessing a plurality of contours of a hard palate of the patient;
engaging the hard palate of the patient at a first desired location;
applying gradual pressure to the first desired location until the hard palate begins to move;
applying constant pressure to the first desired location until the hard palate is repositioned to a desired orientation;
providing a custom fit retainer manufactured to retain the hard palate and soft palate in the desired orientation;
engaging a plurality of teeth of the patient with the retainer;
pressing the retainer against the hard palate and soft palate of the patient; and
wearing the retainer for a prescribed period of time.

15) The method of neuromuscular therapy of claim 14, wherein the constant pressure is in a trajectory towards a nasal cavity of the patient.

16) The method of neuromuscular therapy of claim 14, further comprising the steps of:
engaging the hard palate of the patient at a second desired location of the hard palate;
applying gradual pressure to the second desired location until the hard palate begins to move; and
applying constant pressure to the second desired location.

17) The method of neuromuscular therapy of claim 16, further comprising the steps of:
repeating the steps of engaging new desired locations of the hard palate, applying gradual pressure to the new desired locations until the new desired locations begin to move, and applying constant pressure to the new desired locations until the hard palate is repositioned to a desired orientation.

18) The method of neuromuscular therapy of claim 14, wherein the first desired location is at a midline of the hard palate.

19) The method of neuromuscular therapy of claim 14, wherein the assessment of the contours of the patient's hard palate is achieved by applying a probing pressure to landmarks on the hard palate.
